(19) United States
(12) Patent Application Publication
(10) Pub. No.: US 2021/0241204 A1
(43) Pub. Date: Aug. 5, 2021

(54) PROVIDER CLASSIFIER SYSTEM, NETWORK CURATION METHODS INFORMED BY CLASSIFIERS

(71) Applicant: Embold Health, Inc., Nashville, TN (US)

(72) Inventor: Daniel Stein, Nashville, TN (US)

(21) Appl. No.: 16/782,992

(22) Filed: Feb. 5, 2020

(51) Int. Cl. G06Q 10/06; G16H 50/20; G16H 40/20; G06Q 30/02; G06N 20/00

(52) U.S. Cl. CPC G06Q 10/063112; G16H 50/20; G06N 20/00; G06Q 30/0201; G16H 40/20

(57) ABSTRACT

A method of classifying a provider includes identifying differentiating services from services performed by providers within a selected specialty and selected market, curate a list of differentiating services from the identified differentiating services, mapping at least one differentiating service of the list of differentiating services to one or more coding fields of claims data, analyzing practices of providers within the selected specialty and the selected market, based on the analysis of practices of providers, generating a distribution of a volume of the at least one differentiating service performed by providers within the selected specialty and the selected market, determining a threshold within the distribution of the volume of the at least one differentiating service for classifying a provider as a specialist in performing the at least one differentiating service, and classifying one or more providers as a specialist in performing the at least one differentiating service based on the threshold.

### TECHNICAL FIELD

This disclosure relates generally to a provider classification system for classifying providers as qualifying as specialist and/or subspecialists. This disclosure further relates to curating provider networks based on performance scores of providers within the provider network, wherein the provider networks are refined base on assigned classifiers. This disclosure also related to modifying networks based on the performances scores of the provider within the provider networks.

### BACKGROUND

Directories are lists of providers that are searchable by various criteria, including specialty and sub-specialty. Conventionally, data for fields of the directories identifying (e.g., classifying) the providers as specialists and/or sub-specialists may be self-reported. Additionally, often, the data is acquired (and input into the directories) via web-crawler bots, which download and index website information. Furthermore, the data can be acquired from information included within medical claims. For instance, the medical claims can include fields indicating a specialty (e.g., “Anesthesiologist”).

The foregoing-described directories are sometimes utilized and relied upon when creating and curating provider networks and/or defining a group of providers within a given market as specialists. However, because the sources of the data designating providers as specialists within the directories are often inaccurate and/or incomplete, the directories that rely on these sources may also be inaccurate and/or incomplete in regard to provider specialties and practices. As such, any conclusions (e.g., curated networks) reached relying on the directories may be inaccurate and misleading. For instance, the directories often indicate providers as specialists in particular areas, when the providers are, in fact, not specialists in those areas. Furthermore, directories known to the inventors of this disclosure often fail to recognize sub-specialties due to the sub-specialties not being an option within the directory, not being self-reported by providers, not being indicated online, and/or not being indicatable within medical claims. As a result of the foregoing, sub-specialties may be, if represented, accurately represented in conventional directories known to inventors of this disclosure.

Among the many concerns from inaccurate provider directories, is that patients seeking a specific specialist may visit and/or being cared for providers who do not qualify as the specific specialist. This results in wasted time and money on the patient's part, and can result in the patient receiving inappropriate, overly costly, ineffective, and/or harmful treatment.

### BRIEF SUMMARY

The various embodiments described below provide benefits and/or solve one or more of the foregoing or other problems in the art with systems and methods for definitions of provider specialties and subspecialties. Some embodiments of the present disclosure include a method of method of classifying a provider. The method may include identifying differentiating services from services performed by providers within a selected specialty and selected market; curating a list of differentiating services from the identified differentiating services; analyzing practices of providers within the selected specialty and the selected market; based on the analysis of practices of providers, generating a distribution of a volume of at least one differentiating service performed by providers within the selected specialty and the selected market; determining a threshold within the distribution of the volume of the at least one differentiating service for classifying a provider as a specialist in performing the at least one differentiating service; and classifying one or more providers as a specialist in performing the at least one differentiating service based on the threshold.

Some embodiments of the present disclosure include system comprising: at least one processor; and at least one non-transitory computer-readable storage medium storing instructions thereon that, when executed by the at least one processor, cause the system to: curate a list of differentiating services performed by providers within a selected specialty and a selected market; analyze practices of providers within the selected specialty and the selected market; based on the analysis of practices of providers, generate a distribution of a volume of at least one differentiating service performed by providers within the selected specialty and the selected market; determine a threshold within the distribution of the volume of the at least one differentiating service for qualifying a provider as a specialist in performing the at least one differentiating service; assign each provider within the selected market a percentage ranking within the distribution of a volume of the at least one differentiating service; generate classifiers for one or more providers, each classifier including an indication of the threshold, the provider's percentage ranking, and the specialty in performing the at least one differentiating service; and assign the classifiers to respective one or more providers.

One or more embodiments of the present disclosure include a method of classifying a provider. The method may include analyzing practices of providers within a selected specialty and a selected market by analyzing claims data; based on the analysis of practices of providers, generating a distribution of a volume of at least one differentiating service performed by providers within the selected specialty and the selected market; determining a threshold within the distribution of the volume of the at least one differentiating service for classifying a provider as a specialist in performing the at least one differentiating service; and classifying one or more providers as a specialist in performing the at least one differentiating service based on the threshold.

### BRIEF DESCRIPTION OF THE DRAWINGS

Various embodiments will be described and explained with additional specificity and detail through the use of the accompanying drawings in which:

FIG. 1 illustrates a schematic representation of an environment within which a provider classification system 104 can operate in accordance with one or more embodiments of the present disclosure;

FIGS. 2A and 2B illustrate a sequence flow diagram that a provider classification system 104 can utilize to classify providers in accordance with one or more embodiments;

FIG. 3A-3H depict services (e.g., classifiers) within various subspecialties in accordance with one or more embodiments;

FIG. 4 illustrates a sequence flow diagram that a provider classification system 104 can provide provider information from provider directories generated by the provider classification system 104 according to one or more embodiments of the present disclosure;

FIG. 5 illustrates a sequence flow diagram that a provider classification system 104 can utilize to determine performance scores for providers from a denominator population of classified providers in accordance with one or more embodiments;

FIG. 6 illustrates a sequence-flow diagram showing various acts of a client device and a provider determination system, in accordance with various embodiments of facilitating communications between client devices and the provider determination system;

FIG. 7 illustrates a detailed schematic diagram of a provider classification system 104 according to one or more embodiments of the present disclosure; and

FIG. 8 illustrates a block diagram of an exemplary computing device in accordance with one or more embodiments.

### DETAILED DESCRIPTION

The illustrations presented herein are not actual views of any particular provider classification system, or any component thereof, but are merely idealized representations, which are employed to describe the present invention.

As used herein, the singular forms following “a,” “an,” and “the” are intended to include the plural forms as well, unless the context clearly indicates otherwise.

As used herein, the term “may” with respect to a material, structure, feature, function, or method act indicates that such is contemplated for use in implementation of an embodiment of the disclosure, and such term is used in preference to the more restrictive term “is” so as to avoid any implication that other compatible materials, structures, features, functions, and methods usable in combination therewith should or must be excluded.

As used herein, any relational term, such as “first,” “second,” etc., is used for clarity and convenience in understanding the disclosure and accompanying drawings, and does not connote or depend on any specific preference or order, except where the context clearly indicates otherwise.

As used herein, the term “substantially” in reference to a given parameter, property, act, or condition means and includes to a degree that one skilled in the art would understand that the given parameter, property, or condition is met with a small degree of variance, such as within acceptable manufacturing tolerances. By way of example, depending on the particular parameter, property, or condition that is substantially met, the parameter, property, or condition may be at least 90.0% met, at least 95.0% met, at least 99.0% met, or even at least 99.9% met.

As used herein, the term “about” used in reference to a given parameter is inclusive of the stated value and has the meaning dictated by the context (e.g., it includes the degree of error associated with measure of the given parameter, as well as variations resulting from manufacturing tolerances, etc.).

As used herein, the term “provider” refers generally to a health care provider including any conventional health care provider (e.g., general practitioners (e.g., internal medicine, family practice, emergency room, general surgery doctors, etc.) and/or specialty doctors (e.g., cardiologists, pediatric doctors, obstetricians and gynecologists, optometrists, ophthalmologists, orthopedic surgeons, etc.). The term “provider” also includes physician assistants, nurse practitioners, and other healthcare providers that typically enjoy independent practice rights.

As used herein, the term “domain” may refer to categories of performance scores. For instance, the term “domain” may refer to the categories of appropriateness, effectiveness, and/or cost as performance scores.

As used herein, a provider “specialty” may refer to an occupation, practice area, and/or field of study of a provider such as, e.g., cardiology, pediatrics, gynecology, optometry, ophthalmology, orthopedic surgery, obstetrics, etc.

As used herein, a provider “subspecialty” may refer to an occupation, practice area, and/or field of study of a provider that is part of a broader specialty. For instance, a subspecialist may refer to an expert (e.g., a provider expert) in that part of the broader specialty.

As used herein, the term “provider network” may refer to an electronic database of providers. The provider network may be utilized by insurance companies to determine how much to reimburse a provider for a performed treatment or procedure. For instance, the provider network may help determine whether the treatment or procedure was performed in-network or out-of-network). Accordingly, when a medical claim is received by an insurance company (e.g., a payment processor of the insurance company), the insurance company can decide (e.g., automatically decide) whether the provider is in-network or out-of-network, which in turn, dictates how much is reimbursable to the provider and how much is the patient's responsibility.

As referred to herein, the term “directories” and any derivative terms may include electronic records (e.g., electronic records accessible via the Internet, network-published directories, etc.) that include searchable lists of providers (e.g., doctors). For instance, the electronic records may include fields (e.g., data input areas) that can be utilized to search the directories. For example, the directories may be searchable by users and/or software via one or more of the following: key word searches, sorting by fields, manually viewing the fields, searching tags and/or labels, etc.

As noted above, various embodiments described below provide benefits and/or solve one or more of the foregoing or other problems in the art with systems and methods for definitions of provider specialties and subspecialties, sources for directories, and the directories themselves. For instance, it would be beneficial to have directories (e.g., data sets) that accurately reflect providers' actual practices when curating provider networks and accurately classify providers as specialists based on the actual practices of the providers.

Some embodiments of the present disclosure include a provider classification system for classifying providers as a sub specialist based on the actual practices of the providers. For example, the provider classification system may identify differentiating services within services typically performed within a specialty that differentiate at least some providers from other providers within the specialty. Furthermore, based on a given differentiating service, the provider classification system may identify providers that perform a threshold amount of the differentiating service. Moreover, for providers that perform the threshold amount of the differentiating service, the classification system may classify the providers as subspecialist in performing that differentiating service or as a subspecialist that typically providers that differentiating service.

One or more embodiments of the present disclosure include a provider classification system for determining performance scores of providers based on defined performance measures. For instance, a provider classification system may determine performance scores such as an appropriateness score, an effectiveness score, and/or a cost score. Furthermore, the performance scores may be informed (e.g., refined, improved, etc.) by the classifiers determined herein. For example, a denominator population of providers determined via the classification process may yield more accurate data from which the performance scores can be determined. As such the system of the present disclosure empowers employers, health plans, and health systems to identify top performing providers, top performing care settings, and/or top performing provider-setting pairs.

One or more embodiments of the present disclosure include curating a provider network based on the performance scores of the providers within the provider network. In some embodiments, a provider classification system may curate a provider network within a given and for a given specialty.

Additionally, a provider classification system described herein improves a process of building provider networks. In particular, a provider classification system provides performance scores, informed as described herein by the classification process, so that users can build network to include the highest scoring providers and/or practice groups within a given region and/or within a given specialty. As noted above, the resulting network is informed (e.g., refined and improved) based on the classification process described herein.

The provider classification system of the present disclosure provides several advantages. For instance, the provider classification system remove or reduces any need to visit multiple doctors while trying to find a specialist that actually treats a specific condition (e.g., a rare condition) and/or obtaining a referral. Furthermore, the provider classification system 104 provides more accurate data in regard to which providers qualify as specialists and/or subspecialists. For instance, while a provider may claim to offer a service (e.g., claim to offer service on the provider's website), the provider classification system of the present disclosure may provide a list of providers to a user where the providers in the list actually do perform the service, and the list of providers may include the highest ranked (e.g., curated) providers within a given market. Furthermore, the provider classification system defines denominator populations of providers that actually practice particular sub-specialties, and as such, curating networks of providers within the sub-specialties and based on the denominator population, provides more accurate performance scores and output data. For instance, the provider classification system removes and/or reduces at least some outlier data.

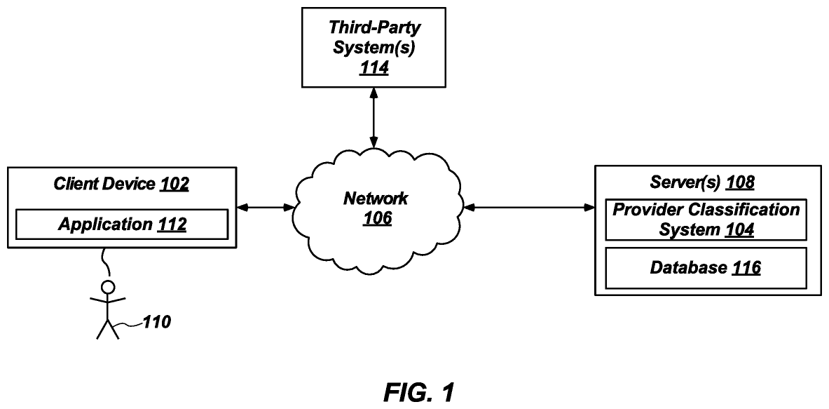

FIG. 1 illustrates a schematic diagram of an environment 100 in which a provider classification system 104 may operate according to one or more embodiments of the present disclosure. As illustrated, the environment 100 includes a client device 102, at least one server 108 including a provider classification system 104, a network 106, and one or more third-party system(s) 114. As used herein, the term “provider classification system 104” refers to a system that classifies providers as specialists and/or subspecialists based on actual practices of the providers. For instance, as will be described in further detail below, a provider classification system 104 may identify differentiating services performed within a given specialty or subspecialty, and upon identifying the differentiating services, the provider classification system 104 may generate a distribution of volume of each differentiating service performed within a given market by providers within that market. Moreover, the provider classification system 104 may classify a provider as a specialist and/or subspecialist if the provider meets some threshold level of providing the differentiating service. For instance, if the provider has performed the differentiating service a threshold number of times, the provider classification system 104 may classify a provider as a specialist and/or subspecialist within the specialty. Conversely, for example, if the provider has not performed the service a threshold number of times, the provider classification system 104 may not classify the provider specialist and/or subspecialist even if the provider is self-identified as the specialist and/or subspecialist. Additionally, upon classifying providers within a given market, the provider classification system 104 may generate searchable directories of providers where the providers are identified based on their respective classification (e.g., sub specialty).

In some embodiments, the client device 102 may include an application 112 (e.g., a tool application) for enabling users to interact with a provider classification system 104 in order to initiate a classification process of providers, query generated provider directories, initiate curation of provider networks, and/or view curated provider networks and/or curated groups of providers (e.g., a specialty within a given market). Furthermore, in some embodiments, the application 112 may enable users to initiate curation of a given provider network, which is refined and improved (e.g., made more accurate) by the classification of the providers. In particular, the client device 102 may execute one or more applications (e.g., application 112) for performing the functions of the various embodiments and processes described herein. For example, in some instances, the application 112 may be web application. In some embodiments, the application 112 may be local to the client device 102. In other embodiments, the application 112 may be stored and/or at least partially operated via a cloud computing service.

In one or more embodiments, the application 112 may be a native application installed on the client device 102. For example, the application 112 may be a mobile application that installs and runs on a mobile device, such as a smart phone or a tablet. The application 112 may be specific to an operating system of the client device 102. Further, the application 112 may be independent of a provider classification system 104. Alternatively, the application 112 may be a client application that is associated with a provider classification system 104 and configured to enable interaction directly with a provider classification system 104 through the application 112.

The provider classification system 104, the client device 102, and the third-party system(s) 114 may communicate via the network 106. In one or more embodiments, the network 106 includes a combination of cellular or mobile telecommunications networks, a public switched telephone network (PSTN), and/or the Internet or World Wide Web and facilitates the transmission of data between the client device 102 and a provider classification system 104. The network 106, however, may include various other types of networks that use various communication technologies and protocols, such as a wireless local network (WLAN), a wide area network (WAN), a metropolitan area network (MAN), other telecommunication networks, or a combination of two or more of the foregoing networks. Although FIG. 1 illustrates a particular arrangement of the client device 102, the server 108, the third-party system(s) 114, and the network 106, various additional arrangements are possible. For example, the server 108 and, accordingly, a provider classification system 104, may directly communicate with the client device 102, bypassing the network 106.

As illustrated in FIG. 1, a user 110 may interface with the client device 102, for example, to communicate with the server 108 and to utilize a provider classification system 104 in any of the manners described herein. The user 110 may be an individual (i.e., human user), a business (e.g., employer), a group, or any other entity. Although FIG. 1 illustrates only one user 110 associated with the client device 102, the environment 100 may include any number of a plurality of users that each interact with the environment 100 using a corresponding client device.

The client device 102 may be any one or more of various types of computing devices. For example, the client device 102 may include a mobile device such as a mobile telephone, a smartphone, a PDA, a tablet, or a laptop, or a non-mobile device such as a desktop or another type of computing device. Additional details with respect to the client device 102 are discussed below with respect to FIG. 12. In some embodiments, a provider classification system 104 may include one or more systems, servers, and/or other devices for performing any of the of the processes and methods described herein. Furthermore, a provider classification system 104 may include and/or have access to one or more databases 116. For example, in some embodiments, a provider classification system 104 may be implemented by a plurality of server devices that store, within the one or more databases, clinical and claims data, provider information and content, insurance information content, user information and content, medical evidence and clinical practice guidelines, and/or defined services, and/or may facilitate querying any of the foregoing information and content to classify providers and generate curated lists of services provided within a provider specialties. As shown, in some embodiments, a provider classification system 104 may include a database wherein the provider classification system 104 stores received claims data (described below), services lists, classifiers, directories of classified providers, analysis algorithms, curated network data, provider data, etc.

The third-party systems 114 may include additional systems that interface with a provider classification system 104. For example, in some embodiments, the third-party systems 114 may include third-party systems having and storing relatively large amounts of clinical and claims data (referred to herein collectively as “claims data”). As used herein, claims data may include records of medical events that are incurred by the patient visiting a healthcare professional (e.g., provider) or a facility in the ambulatory, outpatient, inpatient, home, and other environments where medical services may be delivered. Furthermore, claims data may have a claim line item (CLI) structure, where each claim of the claims data generally includes one or more CLIs. As is also known in the art, claims data may be recorded via various forms of data including client-defined codes, value fields (e.g., input fields), dates, system-specific codes, and standard codes (e.g., current procedural terminology (CPT) codes). In some embodiments, claims data merely includes electronic data that includes information regarding one or more instances of diagnosis, procedures, utilization, and/or combinations thereof and in combination with other things. The information may be organized into fields, but may be organized in other structures such that the fields may include fields, tags, etc.

Furthermore, the third-party systems may include systems having and storing complimentary data sources including standalone claims data, standalone clinical data derived from the electronic health record, linked claims-clinical data, and employer data, to develop, validate, and execute measures. Obtained from payers, individual claims represent transactions between providers and/or facilities and payers. As is described in greater detail below, a provider classification system 104 may also incorporate both structured and unstructured clinical data derived from the electronic health records to supplement information contained in claims to support cohort development and validation, measure development and validation, and system execution.

In some embodiments, the third-party systems 114 may include one or more insurance providers (e.g., Blue Cross Blue Shield), employers sponsoring health plans, provider systems, health care organizations, hospitals, etc. In additional embodiments, the third-party systems 114 may include validations systems (e.g., systems associated with clinical experts and groups) for validating at least portions of a classifying process (e.g., classifiers, statistical models, defined thresholds, etc.) of a provider classification system 104. As is described in further detail below, a provider classification system 104 may utilize claims data to curate lists of services performed by providers within specialties and/or sub specialties and to determine providers' actual practices (e.g., services actually provided by providers as evidenced in claims data).

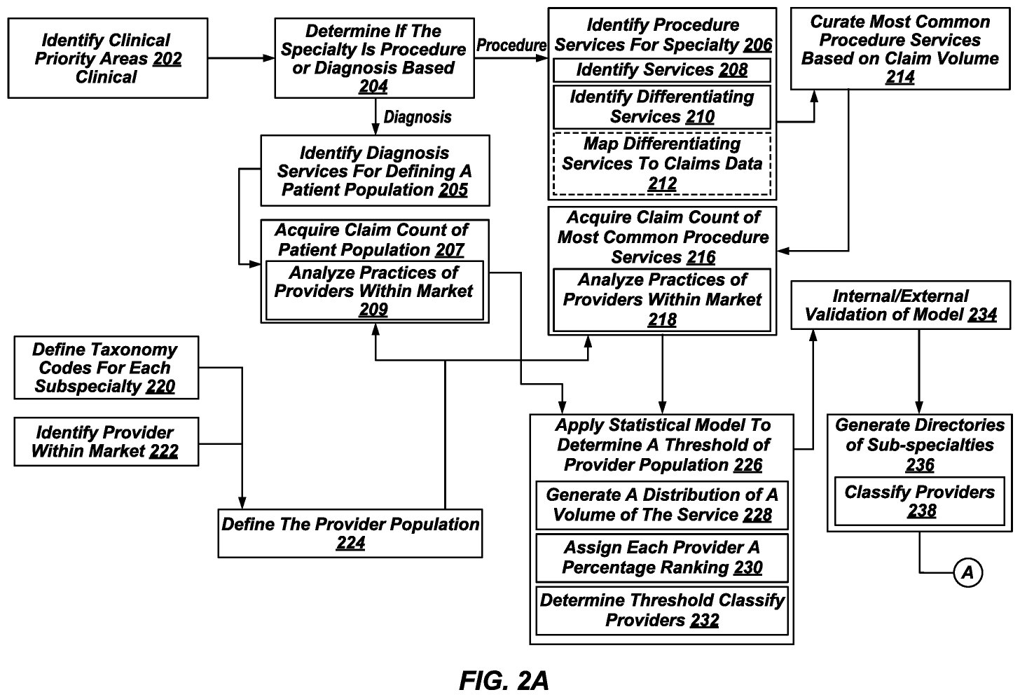

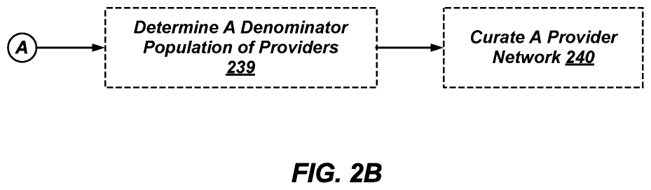

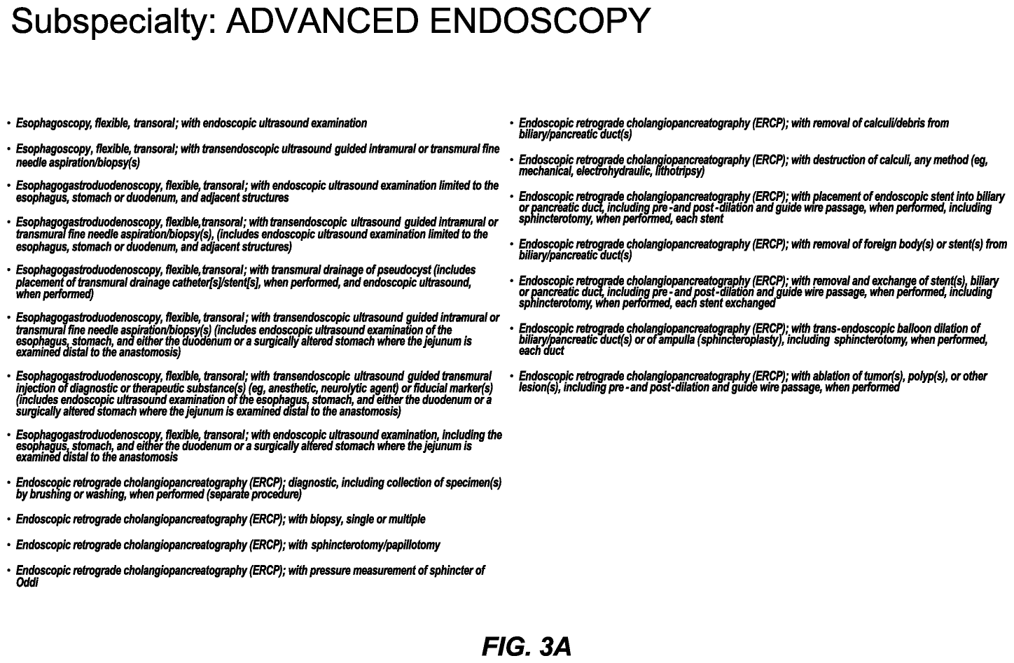

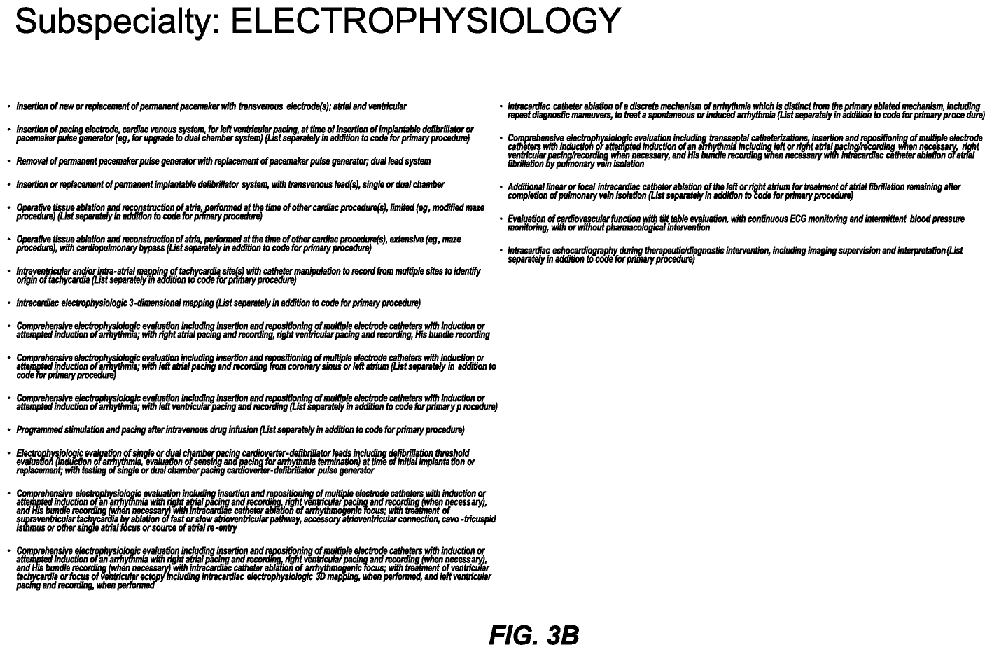

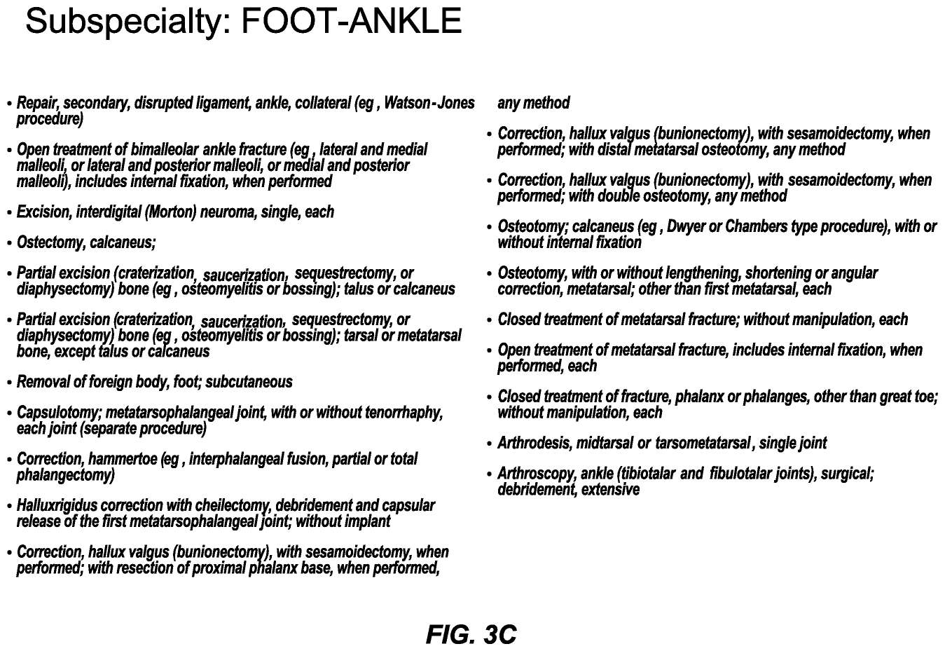

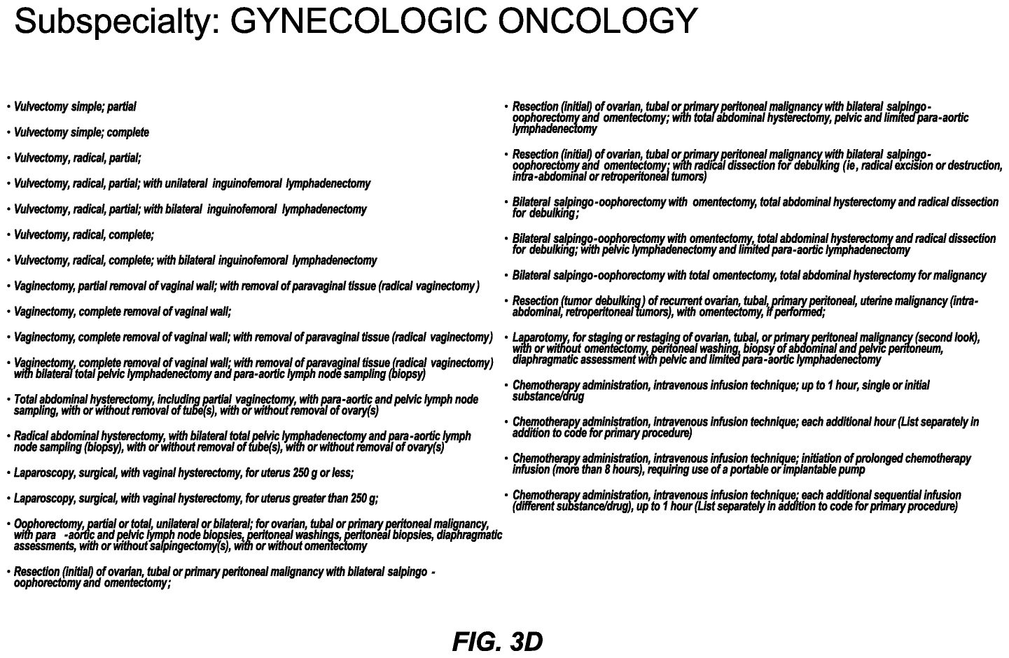

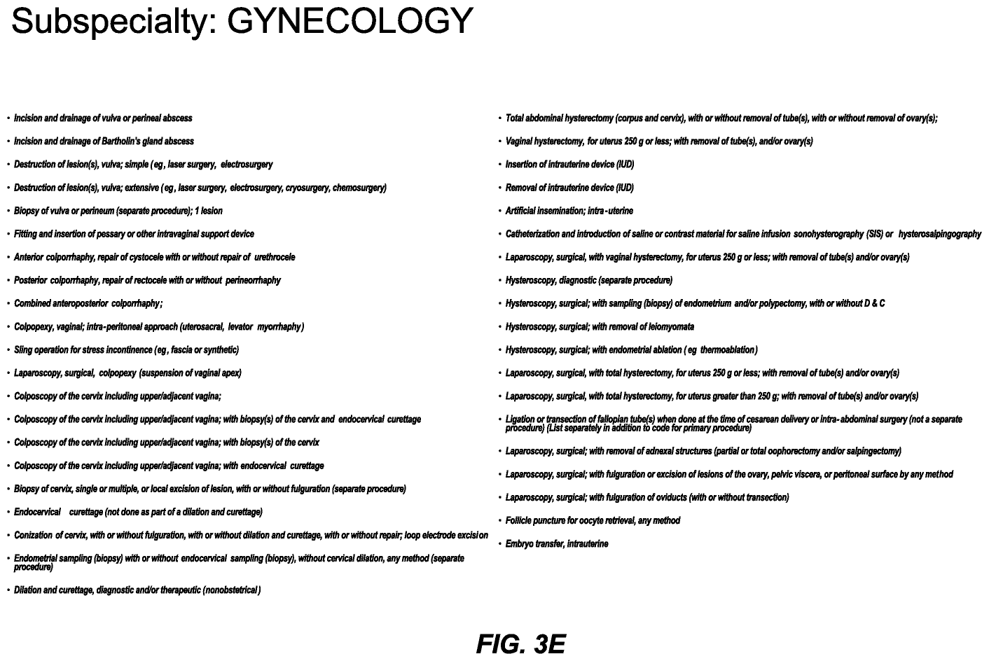

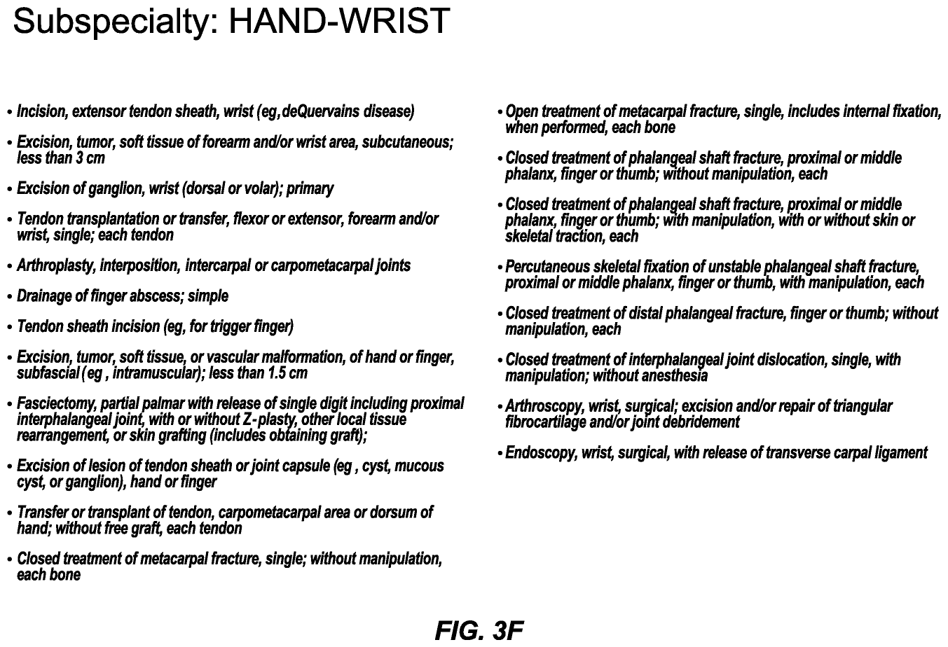

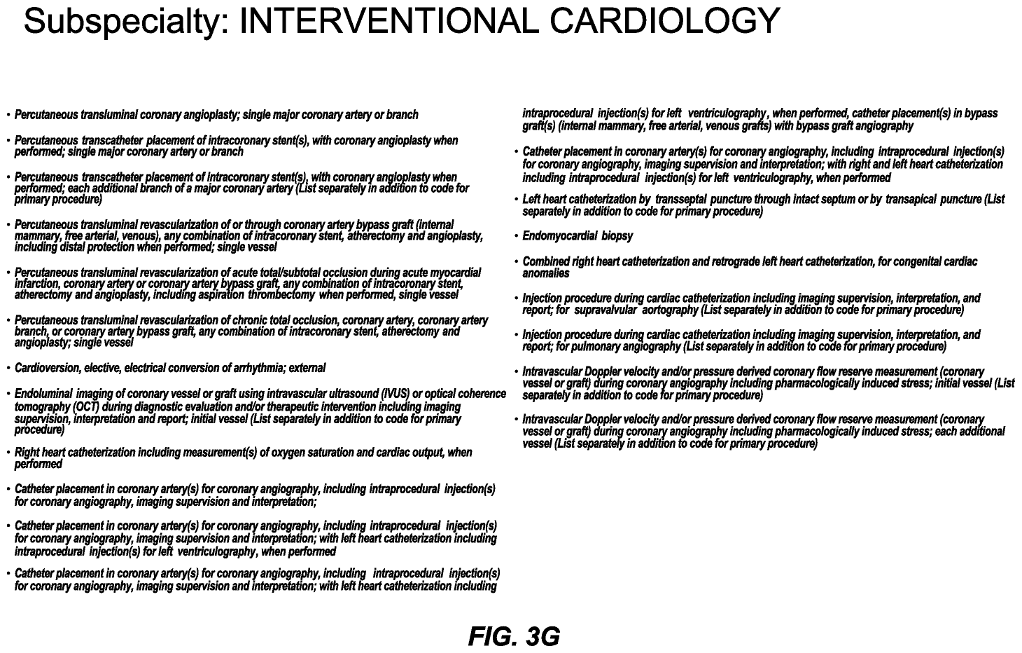

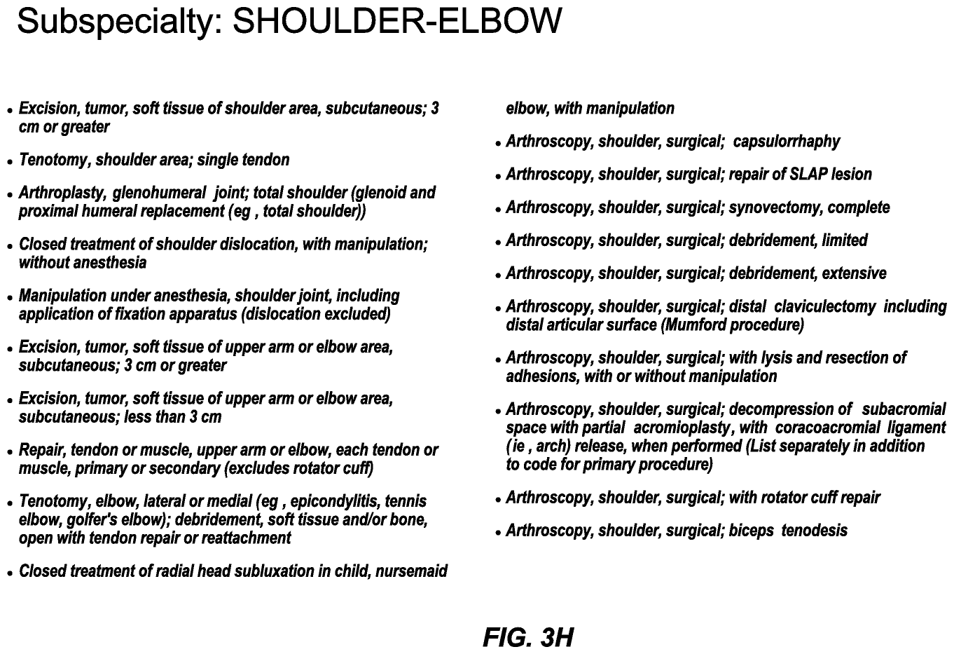

FIGS. 2A and 2B illustrates a sequence-flow diagram showing a method 200 including various acts of a provider classification system 104 for determining curated lists of services performed within a provider specialty and classifying providers within a market as specialists and/or subspecialists based on actual services performed by the providers. The lists of services (e.g., classifiers) are discussed in greater detail below in regard to FIGS. 3A-3H. The provider classification system 104 described in regard to FIGS. 2A and 2B may be example embodiments of a provider classification system 104 described in regard to FIG. 1.

As shown in act 202 of FIG. 2A, in some embodiments, a provider classification system 104 may identify clinical priority areas (e.g., clinical specialties). As used herein, a “clinical area” may refer to a clinical category and/or patient condition and treatment plan within an area of practice typically performed and overseen by a provider, as defined above. For example, in some embodiments, a clinical area may include a specialty such as low-risk obstetrics, orthopedic surgery (joint care), orthopedic surgery (spinal care), cardiology, primary care, gastroenterology, pulmonology, or any other specialty. Furthermore, although specific specialties are described herein, one of ordinary skill in the art will readily recognize that the clinical priority areas may include any specialty in which providers practice. In some embodiments, identifying clinical priority areas may include identifying clinical areas that typically include key-clinical decision points (e.g., whether or not to recommend surgery) in advance of high-cost procedures and/or high-morbidity procedures such as arthroplasty, spinal laminectomy/fusion, cardias stenting, C-section, etc. In some embodiments, identifying clinical priority areas may include identifying clinical priority areas based on subject matter expert input data, clinical literature, input from the clinical Advisory Board, etc. For instance, in some embodiments, the clinical priority areas may be user-defined by employers, provider networks, individuals (e.g., providers and/or clinical experts), etc. In view of the foregoing, a provider classification system 104 may identify areas within medical practices that typically result in key-clinical decision points that have relatively significant consequences.

In response to identifying clinical priority areas and specialties and/or subspecialties within those clinical priority areas, a provider classification system 104 determines whether the specialties (or subspecialties) are procedure or diagnosis based specialties, as shown in act 204 of FIG. 2A. For instance, the provider classification system 104 may determine a type of services typically provided within the specialties. For instance, the provider classification system 104 may determine whether a majority of the services typically provided within a given specialty are procedure based or are diagnosis based. In some embodiments, the provider classification system 104 determines the types of services typically performed based on one or more of an analysis of claims data, subject matter expert input data, clinical literature, input from the clinical Advisory Board, definitions of the National Uniform Claim Committee, etc.

If a provider classification system 104 determines that the given specialty is procedure based, the provider classification system 104 identifies procedure services and/or related procedure codes typically performed within the given specialty, as shown in act 206 of FIG. 2A. In some embodiments, identifying procedure services for the given specialty includes 1) identifying services (e.g., procedures, treatments, etc.) that are typically performed by providers identified within the given provider specialty, as shown in act 208 of FIG. 2A, 2) identifying differentiating services from the identified services, as shown in act 210 of FIG. 2A, and 3) in some embodiments, mapping the differentiating services to claims data. In one or more embodiments, the given provider specialty may be defined by and/or based on definitions from the National Uniform Claim Committee (NUCC), American Board of Medical Specialties (ABMS), American Osteopathic Association (AOA), CMS, and/or any other entity providing specialty definitions. In other words, which services, and as a result, which procedure codes within claims data, are included within a given specialty may be based on traditional categorizations. Furthermore, the services may be at least partially based on self-identified providers. As a non-limiting example, the provider classification system 104 may identify services that are typically performed by providers identified as a cardiologist, dermatologist, gastroenterologist, etc.

In some embodiments, the services may be identified from publically available lists, known data sources, subject matter expert input data, clinical literature, input from the clinical Advisory Board, coding guidelines from organizations such as American Society for Gastrointestinal Endoscopy, etc. Furthermore, as will be described in greater detail below, in some embodiments, the services may be identified and/or refined based on claims data, which may be analyzed by a provider classification system 104. In some embodiments, the services may be related to procedures or practices for treating specific conditions. Potential services identified by a provider classification system 104 are described in greater detail in regard to FIGS. 3A-3H. In some embodiments, a provider classification system 104 may identify a list of services typically performed by providers within a given defined provider specialty or subspecialty.

As noted above, the provider classification system 104 also identifies differentiating services from the identified services, as shown in act 210 of FIG. 2A. For example, a provider classification system 104 may identify differentiating services within the identified services that cause providers within a given specialty or subspecialty to be differentiated from each other based on the performed services. In some embodiments, a provider classification system 104 may identify the differentiating services based on an analysis of claims data and a threshold percentage of providers performing or not performing that service. In other words, a provider classification system 104 may identify services that are not offered globally (i.e., by each provider) within a specialty or subspecialty. Furthermore, a provider classification system 104 may identify services that that are not offered uniformly across a specialty or subspecialty. For instance, one provider may perform the service fifty times per year, and other providers may have only performed the service three to five times per year. As a non-limiting example, a service that 100% of providers within the given provider specialty perform would not qualify as a differentiating service, as it yields no differentiating information between providers. As another non-limiting example, a service that 75% of providers within the given specialty perform provides differentiating information between providers. As noted above, in some embodiments, the differentiating services may be identified at least partially by analyzing claims data to identify services that meet particular threshold percentiles. For instance, in some embodiments, the differentiating services provide differentiating information (e.g., identifies differences) of at least 30% of the population relative to the other 70%. In other embodiments, differentiating services provide differentiating information (e.g., identifies differences) of at least 25%, 20%, 15%, 10%, 5%, or any other percentage (or percentile value) of the population relative to a remainder of the population. In additional embodiments, the differentiating services may be identified at least partially based on subject matter expert input data, clinical literature, input from the clinical Advisory Board, coding guidelines from organizations such as American Society for Gastrointestinal Endoscopy, etc. In yet further embodiments, there may not be a discrete threshold. In other words, the threshold may be variable (e.g., flexible). For instance, the threshold may be at least partially determined to increase sensitivity for identifying subspecialists (described herein) (e.g., reduce the likelihood of false negatives) and/or increase specificity for identification of subspecialists (e.g., reduce likelihood of false positives). In view of the foregoing, the threshold may be at least partially determined by end results.

In some embodiments, as mentioned above, identifying the procedure services for each specialty may further includes mapping each differentiating service to claims data, as shown in act 212 of FIG. 2A. In some embodiments, a provider classification system 104 may (e.g., associate) the differentiating services to coding fields (e.g., current procedural terminology (“CPT”) codes (e.g., the national coding set for physician and other health care professional services and procedures)) of claims data. As is known in the art, the CPT evidence-based codes accurately encompass the full range of health care services. Furthermore, as is known in the art, the CPT code descriptors are clinically focused and utilize common standards so that a diverse set of users can have common understanding across the clinical health care paradigm) of claims data. In other words, a provider classification system 104 associates the differentiating services to coding fields of claims data by generating a respective algorithm (i.e., specifications of the selected differentiating services) for each differentiating service. Put another way, a provider classification system 104 specifies the differentiating services in terms of claims data as the algorithm. For example, a provider classification system 104 defines the differentiating services as claims-based, differentiating services. Furthermore, a provider classification system 104 may map the differentiating services to any forms of data typically found within claims data (e.g., the forms of data described above in regard to FIG. 1) via the above-described manner. In view of the foregoing, a provider classification system 104 may map the differentiating services to data within claims data that indicates whether or not the service was performed by a provider.

In some embodiments, a provider classification system 104 may map the differentiating services to coding fields and/or other forms of data within the claims data via conventional data mapping methodologies. For example, a provider classification system 104 may map the differentiating services to coding fields and/or other forms of data within the claims data via field mapping and field value mapping methodologies. In field mapping, fields in the claims data are identified that correspond to each of the required fields. In field value mapping, for each field where applicable, a provider classification system 104 determines the field value that corresponds to each possible value that may occur in the claims data, mapped to a differentiating service. In some embodiments, a provider classification system 104 may map the differentiating services to coding fields and/or other forms of data within the claims data by creating eXtensible Stylesheet Language Transformations (XSLT Transform, by simultaneously evaluating actual data values of the coding fields of the claims data (e.g., data sources) using heuristics and statistics to automatically discover complex mappings between A) the differentiating services and B) the coding fields and/or other forms of data within the claims data. In some embodiments, the foregoing methodologies are used to determine transformations between A) the differentiating services and B) the coding fields and/or other forms of data within the claims data via discovering substrings, concatenations, arithmetic, case statements as well as other kinds of transformation logic. The foregoing methodologies may also determine data exceptions that do not follow the discovered transformation logic. In one or more embodiments, mapping the differentiating services to coding fields may include semantic mapping.

As a non-limiting example, a provider classification system 104 may identify Ostectomy, calcaneus as a differentiating service within a specialty of podiatry, and the provider classification system 104 may map the differentiating service, Ostectomy, calcaneus, to coding fields (e.g., CPT codes, ICD-10 codes, and/or input fields) of the claims data that may indicate that a provides the differentiating service. Furthermore, a provider classification system 104 may define the mapped coding fields as at least a portion of a specification of the differentiating service. For instance, the mapping data may comprise at least a portion of a data packet defining the differentiating service in terms of claims data.

In other embodiments, the differentiating services are defined in terms of claims codes upon identification. For instance, the services typically performed in a specialty may be identified via CPT codes ICD-10 codes, and/or input fields, and no specific mapping procedures are necessary. However, the differentiating services may be associated within the CPT codes within the provider classification system 104.

In response identifying procedure services (e.g., differentiating services) within a given specialty, a provider classification system 104 curates the most common procedure services for the given specialty, as shown in act 214 of FIG. 2A. For example, the provider classification system 104 may curates the top 20, 30, 50 or any other number of the most common differentiating services performed within a selected market as evidenced by claims data. In other embodiments, a provider classification system 104 may curate (i.e., rank) the differentiating services based on the most differentiating services performed within the specialty while still meeting the thresholds discussed above in regard to act 210 of FIG. 2A. For instance, a least common performed differentiating service and/or a service performed in majority by a sub group of the specialty or the subspecialty may be identified as a most differentiating service, and the differentiating services may be curated accordingly. In further embodiments, the provider classification system 104 curates the most common differentiating services for the given specialty to minimize noise between, for example, generalists and specialists.

Returning to act 204 of FIG. 2A, if a provider classification system 104 determines that the specialty is diagnosis based, the provider classification system 104 identifies diagnosis services (e.g., ICD-10-CM codes) of the specialty to define a patient population (e.g., a patient population matching the diagnosis codes), as shown in act 205 of FIG. 2A. The diagnosis services may be identified via any of the manners described above in regard to acts 206-212. In some embodiments, based on the diagnosis services typically provided within the given specialty and within a given market, the provider classification system 104 may define a patient population receiving the diagnosis services of the given specialty or subspecialty within the given market. For example, the provider classification system 104 may identify the patient population by analyzing claims data of the providers within the given specialty or subspecialty and the given market. Additionally, in one or more embodiments, the provider classification system 104 may curate the most common diagnosis services via any of the manners described above in regard to act 214 of FIG. 2A.

Referring to acts 205 and 214 together, upon curating the most common services (e.g., procedure services) and/or identifying services (e.g., diagnosis services) to define a patient population, a provider classification system 104 may ascertain (e.g., acquire, determine, identify, etc.) a claim count of the most common procedure services and/or a claim count of the patient population of the diagnosis services, as shown in acts 216 and 207 of FIG. 2A, respectively. For instance, the provider classification system 104 may ascertain a volume of the procedure services provided by each provider within the given market and/or a volume of the diagnosis services provided to a target patient population within the given market, as shown in acts 218 and 209 of FIG. 2A, respectively.

In some embodiments, acquiring the claim count of the most common procedure services and the claim count of the patient population (e.g., diagnosis services) may include analyzing practices of providers identified within a specialty, as shown in acts 218 and 209 of FIG. 2A. Identifying providers within a specialty and/or subspecialty and within a given market is discussed below in regard to acts 220-224 of FIG. 2A. For purposes of clarity and facilitating description herein, the procedure services and the diagnosis services are referred to hereinafter collectively as “differentiating services.” In some embodiments, a provider classification system 104 may analyze practices of providers via claims data. In one or more embodiments, a provider classification system 104 may analyze practices of providers within a specialty or subspecialty and within a given market (e.g., a market selected by a user and/or a market within a provider network). As noted above, in some embodiments, the specialty or subspecialty (and/or providers within the specialty) may be at least partially defined and identified by the NUCC. For example, a provider classification system 104 may analyze claims data associated within each provider matching the specialty or subspecialty and falling within the given market to determine whether the provider performs the differentiating service. A provider classification system 104 may query the claims data according to the map data described herein to determine whether a provider performed the differentiating service. Furthermore, a provider classification system 104 may analyze claims data to determine how often the provider performs the differentiating service. In some embodiments, a provider classification system 104 may determine a number of times a given provider performed the differentiating service within a given period of time (e.g., within the last year, two years, three years, etc.)

In some embodiments, the provider classification system 104 may analyze practices of providers and/or perform any of the analyses described herein via one of more of structured or unstructured machine-learning models. The machine-learning models may include a quadratic regression analysis, a logistic regression analysis, a support vector machine, a Gaussian process regression, ensemble models, or any other regression analysis. Furthermore, in yet further embodiments, the machine-learning models may include decision tree learning, regression trees, boosted trees, gradient boosted tree, multilayer perceptron, one-vs-rest, Naïve Bayes, k-nearest neighbor, association rule learning, a neural network, deep learning, pattern recognition, or any other type of machine-learning.

Referring now to acts 220-224, the method 200 includes defining taxonomy codes for each specialty and/or subspecialty, as shown in act 220 of FIG. 2A. As is known in the art, taxonomy codes are administrative codes for identifying a provider type and area of specialization for health care providers. Typically each taxonomy code is a unique ten character alphanumeric code that enables providers to identify their specialty at the claim level. Therefore, in some embodiments, a provider classification system 104 may define the taxonomy codes for each specialty and/or subspecialty based on conventional taxonomy codes.

Additionally, the method 200 includes identifying providers within a given market, as shown in act 222 of FIG. 2A. For instance, a provider classification system 104 may identify providers within a given geographical area and/or insurance network by acquiring data from public sources, known directories, and/or claims data.

Based on the defined taxonomy codes for a given specialty and/or a subspecialty and the identified providers within a given market, a provider classification system 104 defines a provider population, as shown in act 224 of FIG. 2A. For instance, the provider classification system 104 may categorize providers being associated with the defined taxonomy codes and falling within the given market as the provider population. As discussed above in regard to acts 218 and 209, a provider classification system 104 may analyze the practices of the providers within the defined provider population (e.g., may acquire claim counts from the provider population).

Upon acquire claim counts of the differentiating services and/or the patient population, as discussed above in regard to acts 216 and 207, a provider classification system 104 applies a statistical model to the acquired claim counts to determine a threshold (e.g., a threshold level) of the provider population for identifying (e.g., qualifying) a provider as a specialist and/or subspecialist, as shown in act 226 of FIG. 2A. For example, a provider classification system 104 may apply one or more threshold models to the acquired claim counts. In some embodiments, a provider classification system 104 may apply one or more segmented regression analyses to the acquired claim counts. As a non-limiting example, a provider classification system 104 may apply one or more of deterministic recursive multivariate models or nonlinear autoregressive models to the acquired claim counts to identify a threshold. In some embodiments, act 226 may include generating a distribution of a volume of the differentiating service provided within a given market within a given period of time, as shown in act 228 of FIG. 2A. Additionally, in one or more embodiments, act 226 may include assigning each provider a percentage ranking within the distribution, as shown in act 230 of FIG. 2A. Moreover, act 226 may include determining the threshold value for classifying a provider as a specialist and/or subspecialist within the distribution, as shown in act 232 of FIG. 2A. Each of the foregoing is described in detail below.

As noted above, act 226 may include generating a distribution of a volume of the differentiating service provided within a given market within a given period of time, as shown in act 228 of FIG. 2A. For instance, within the given market, the provider classification system 104 may determine a total number of times the differentiated service was performed within the market. Furthermore, the distribution of a volume of the differentiating service may be distributed according to providers. For instance, each provider within the given market and matching the given specialty may be represented and identified within the distribution. For example, the distribution may indicate the individual statistics of each provider (e.g., how many times the provider performed the service, when, and how often).

As mentioned above, in one or more embodiments, act 226 may include assigning each provider a percentage ranking within the distribution, as shown in act 230 of FIG. 2A. For instance, in response to generating the distribution of a volume of the differentiating service provided within a given market, a provider classification system 104 assigns each provider within the given market a percentage ranking. For example, if a median number times that the differentiating service has been performed by the providers within the market is seventy times, and a given provider performed the differentiating service seventy times, a provider classification system 104 may assign the given provider a 50^{th} percentile ranking. Accordingly, the given provider is ranked relative to the given provider's peers (e.g., the identified provider population). Furthermore, the percentage ranking may be specific to the differentiating service (e.g., associated with the differentiating service). Therefore, the provider classification system 104 may assign a percentile ranking for each differentiating service for each provider.

As also mentioned above, act 226 may include determining the threshold value for classifying a provider as a specialist and/or subspecialist within the distribution, as shown in act 232 of FIG. 2A. In some embodiments, the provider classification system 104 determines a threshold in a curve defined by the distribution of the volume of the differentiating service provided within a given market as qualifying a provider as a subspecialist and/or specialist for providing that differentiating service. For instance, a provider classification system 104 may identify a percentile within the distribution as the threshold. In some embodiments, a provider classification system 104 may identify a characteristic of the curve defined by the distribution as the threshold. For instance, a provider classification system 104 may identify when the curve achieves a particular slope and/or change in slope as the threshold. For instance, a provider classification system 104 may identify a percentile most proximate to the identified characteristics and may determine the percentile to the threshold. In other embodiments, a provider classification system 104 may identify when the curve changes from generally linear to generally exponential as the threshold. Although specific characteristics are described herein, the disclosure is not so limited, and a provider classification system 104 may identify any characteristic of a curve (defined by the distribution) as the threshold.

Upon determining the threshold for identifying a provider as a subspecialist and/or specialist for performing the differentiating service, a provider classification system 104 performs an internal and/or external validation of the statistical model and/or determined threshold, as shown in act 234 of FIG. 2A. For instance, a provider classification system 104 may validate the statistical model and/or determined threshold against conventional specialty and subspecialty definitions stored by a provider classification system 104 within the database 116 of a provider classification system 104. For example, a provider classification system 104 may determine whether the statistical model and/or determined threshold yield specialty and/or subspecialty populations that include providers that self-identify as specialists and/or subspecialists with a defined accuracy. For instance, a provider classification system 104 may determine whether the yield accurate results.

In some embodiments, a provider classification system 104 may validate the statistical model and/or determined threshold against known thresholds. For instance, a provider classification system 104 may validate the statistical model and/or determined threshold against thresholds known to yield accurate specialty and subspecialty populations. In one or more embodiments, validating the initial performance measure may include an iterative process of validating the statistical model and/or determined threshold against known specialty and/or subspecialty populations. In some embodiments, a provider classification system 104 may determine whether the statistical model and/or determined threshold correctly yield accurate specialty and/or subspecialty populations within a particular percentage. For instance, a provider classification system 104 may determine whether the statistical model and/or determined threshold cause a provider classification system 104 to correctly identify specialty and/or subspecialty populations at an accuracy of 80%, 90%, 95%, or 99%. In some embodiments, the statistical model and/or the determined threshold may be verified manually (e.g., via a manual quality assurance process). For instance, the statistical model and/or the determined threshold (e.g., a provider practice) may be verified via phone calls and web scraping or crawling and/or any other manual process for verifying results.

Upon performing the initial validation of the initial performance measure, a provider classification system 104 may provide for external clinical validation of the statistical model and/or determined threshold. For instance, in some embodiments, a provider classification system 104 may send the specifications of the statistical model and/or determined threshold to one or more third-party systems for critique. As a non-limiting example, a provider classification system 104 may send the specifications of the statistical model and/or determined threshold to one or more validation systems (e.g., systems associated with clinical experts and groups), physicians, clinical experts, clinical review boards, etc. for validating the specification of the initial performance measure.

Upon validating the statistical model and/or the threshold, a provider classification system 104 generates one or more searchable directories designating providers as specialists and/or sub specialists, as shown in act 236 of FIG. 2A. For example, a provider classification system 104 may generate directories specific to subspecialties based on the above analysis. In some embodiments, the directories may further include contact information related to the providers (e.g., address, phone number, website, etc.) such that the directories may be searchable and may yield, upon being queried, contact information to a user. In some embodiments, generating the one or more searchable directories may include classifying providers meeting the threshold as a subspecialist and/or specialist for performing the differentiating service, as shown in act 238 of FIG. 2A. For instance, a provider classification system 104 assigns a classifier to the provider, where the classifier indicates that the provider performs the service but also indicates that the provider is a specialist relative to the provider's peers (e.g., the population of the traditionally defined specialty). For example, the classifier is informative of both the fact that the provider performs the differentiating service but also a ranking relative to the provider's peers. In some embodiments, a provider classification system 104 may store one or more data packets within a database associating the classifier within the provider.

In some embodiments, acts 202-238 of FIG. 2A may be in whole or partially performed via one or more machine learning processes. For example, in some embodiments, a provider classification system 104 may analyze claims data and/or any other data indicating actual practices of providers utilizing one or more of regression models (e.g., a set of statistical processes for estimating the relationships among variables), classification models, and/or phenomena models. Additionally, the machine-learning models may include a quadratic regression analysis, a logistic regression analysis, a support vector machine, a Gaussian process regression, ensemble models, or any other regression analysis. Furthermore, in yet further embodiments, the machine-learning models may include decision tree learning, regression trees, boosted trees, gradient boosted tree, multilayer perceptron, one-vs-rest, Naïve Bayes, k-nearest neighbor, association rule learning, a neural network, deep learning, pattern recognition, or any other type of machine-learning.

For example, the provider classification system 104 may apply one or more of the above-described machine learning techniques to claims data and/or any other data indicating actual practices of providers in conjunction with previously identified and/or verified specialists and/or subspecialists. As a non-limiting example, the provider classification system 104 may utilize previously identified and/or verified specialists and/or subspecialists providers to train the machine-learning models to develop definitions of specialties and/or subspecialties based on practices of providers relative to other providers within a traditional specialty and match the claims data (e.g., services performed by providers relative to a population) with the developed specialties and/or subspecialties (i.e., classifications). In other words, via the machine learning model techniques, the provider classification system 104 may learn trends and correlations for developing definitions of specialties and subspecialties and correlations between claims data (e.g., services performed by providers relative to a population) and the developed specialties and/or subspecialties (i.e., classifications). Put another way, the provider classification system 104 may learn the relationship between practices of providers and what constitutes a specialty and/or subspecialty and relationships the claims data (e.g., services performed by providers relative to a population) and the developed specialties and/or subspecialties (i.e., classifications). For example, as will be understood in the art, for a given set of input values (e.g., data indicating actual practices of providers)) of claims data, the provider classification system 104 is expected to produce the same output values (i.e., correct classification of the providers) as would be actually understood by a human operator. In particular, the machine learning models may be trained via supervised learning, as is known in the art. After a sufficient number of iterations, the machine learning models become trained machine-learning models. In some embodiments, the machine learning models may also be trained on historical data (e.g., claim data) from previously identified classifications related to the operations of the provider classification system 104. In view of the foregoing, in some embodiments, the provider classification system 104 may classify providers at least partially via any of the machine-learning techniques described herein.

In some embodiments, the method 200 may optionally include determining a denominator population of providers qualifying as a specialist and/or subspecialist for further analysis, as shown in act 239 of FIG. 2B. For instance, the provider classification system 104 may define a denominator population for a given subspecialty as the providers qualifying as a subspecialist according to the classification process described herein. For example, the denominator population for a given subspecialty may include the providers listed in the generated directory specific to the subspecialty. Determining a denominator population that is refined (e.g., informed and filtered) based on the classifying process described above in regard to acts 202-238 of FIG. 2A may result in better data and more accurate results from subsequent analyses. For example, the method 200 may optionally include determining a denominator population of providers for subsequent analyses such as any of the analyses described in U.S. application Ser. No. 16/584,044, to Stein, filed Aug. 22, 2019, and Titled: PROVIDER ASSESSMENT SYSTEM, METHODS FOR ASSESSING PROVIDER PERFORMANCE, METHODS FOR CURATING PROVIDER NETWORKS BASED ON PROVIDER PERFORMANCE, AND METHODS FOR DEFINING A PROVIDER NETWORK BASED ON PROVIDER PERFORMANCE, the disclosure of which is incorporated in its entirety by reference herein. For example, a provider classification system 104 or the provider classification system 104 in combination with other systems may apply performance measures to the claims data associated with the denominator population to determine measure scores and ultimately determine composite performance scores of one or more providers relative to the denominator population. Furthermore, because the denominator population is informed based on the classification process described herein, the resulting measure scores and composite performance scores may be more accurate because the providers are compared (in the scoring process) to other providers that practice in similar manners (e.g., qualify as a same type of specialist and/or subspecialist as the provider based on practice). Moreover, utilizing the denominator population in comparison to conventional definitions of specialties and/subspecialties may remove and/or reduce outlier data.

In some embodiments, the method 200 may optionally include curating a provider network based at least partially on the denominator population and determined composite scores, as shown in act 240 of FIG. 2B. For instance, a provider classification system 104 or the provider classification system 104 in combination with other systems may curate a provider network or portion thereof via any of the manners described in U.S. application Ser. No. 16/584,044, to Stein, filed Aug. 22, 2019. Curating a provider network utilizing the refined data (e.g., denominator population) may result in more accurate curated provider networks that better reflect actual practices of the providers analyzed therein and may reduce outlier effects. Furthermore, having better curated provider networks may result in better care for patients (e.g., more appropriate care, higher quality of care, and more fair health care costs). Moreover, in view of the foregoing, in some embodiments, a provider classification system 104 may further identify changes in a practice of an individual provider and/or trends of changes in a given specialty and/or subspecialty in terms of practices.

FIGS. 3A-3H list example differentiating services within a plurality of example subspecialties that may be utilized to determine a distribution of volume of the services performed within a market, to classify providers as subspecialists and/or specialists, and to build directories of providers based on classifiers. In particular, FIG. 3A shows the subspecialty of advanced endoscopy and related example differentiating services. FIG. 3B shows the subspecialty of electrophysiology and related example differentiating services. FIG. 3C shows the subspecialty of foot ankle and related example differentiating services. FIG. 3D shows the subspecialty of gynecologic oncology and related example differentiating services. FIG. 3E shows the subspecialty of gynecology and related example differentiating services. FIG. 3F shows the subspecialty of hand-wrist and related example differentiating services. FIG. 3G shows the subspecialty of interventional cardiology and related example differentiating services. FIG. 3H shows the subspecialty of shoulder-elbow and related example differentiating services.

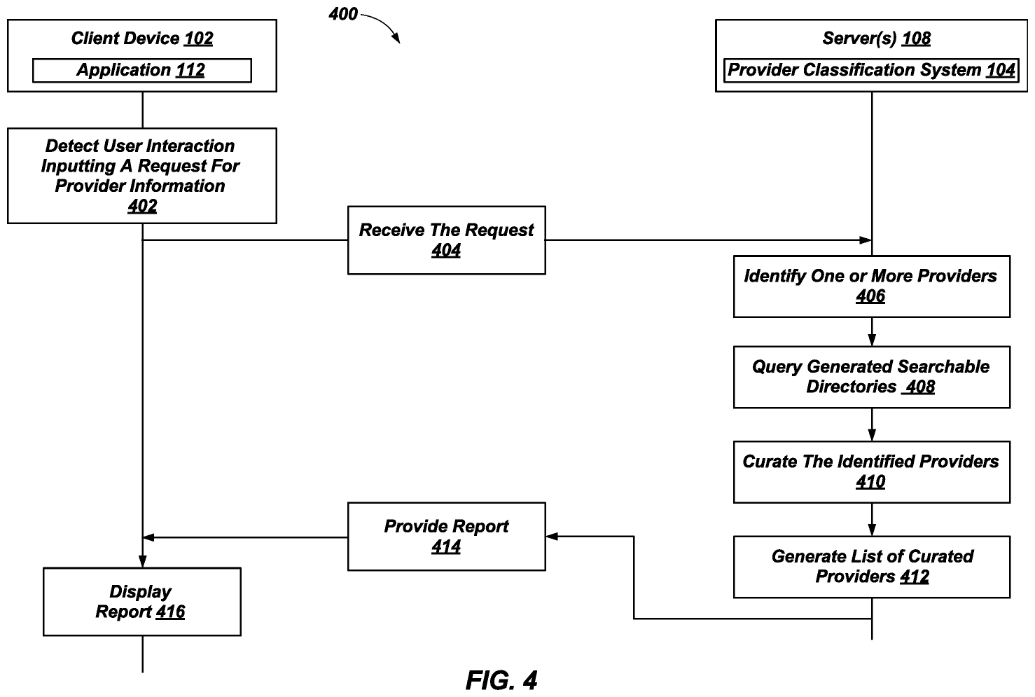

FIG. 4 illustrates a sequence-flow diagram 400 showing various acts of a client device 102 and a provider classification system 104, in accordance with various embodiments of facilitating communications between client devices and the provider classification system 104. The client device 102 and the provider classification system 104 shown in FIG. 4 may be example embodiments of the client device 102 and the provider classification system 104(s) 104 described in regard to FIGS. 1 and 2.

As shown in act 402 of FIG. 4, in some embodiments, the application 112 and/or client device 102 detects a user interaction inputting a request for information regarding a provider and/or a list of providers that treat a particular condition, provide a particular service (e.g., any of the services described above in regard to FIGS. 1-3H), and/or qualify as a particular specialist and/or subspecialist according to the classification process described herein. As used herein, the terms “user interaction” mean a single interaction, or combination of interactions, received from a user by way of one or more input devices (e.g., a touch screen display, a keyboard, a mouse, microphone, camera, etc.) of the client device 102. Furthermore, the user interaction may include one or more of clicking, tapping, or otherwise selecting elements (e.g., letters and/or characters) to include in the message, speaking, and/or capturing video. For example, the application 112 of the client device 102 detects a user interaction inputting a condition and/or desired service within the application 112 in response to the prompt. For instance, the detected user interaction may be made via one or more graphical user interfaces of the application 112. In some embodiments, the request may be limited to a particular market (e.g., geographical region and/or insurance network).

In response to the client device 102 detecting a user interaction inputting the request, a provider classification system 104 receives the request from the client device 102, as shown in act 404 of FIG. 4. For instance, the provider classification system 104 may receive a data package including the request. In some embodiments, a provider classification system 104 may receive a data package via any of the networks described above in regard to FIG. 1.

Upon receiving the request, a provider classification system 104 identifies one or more providers that satisfy the request, as shown in act 406 of FIG. 4. In some embodiments, identifying one or more providers that satisfy the request may optionally include any of the acts described above in regard to acts 202-238 of FIG. 2A. For example, identifying one or more providers that satisfy the request may initiate acts 202-238 of FIG. 2A. Accordingly, in some embodiments, identifying one or more providers that satisfy the request may optionally include identifying services that are typically performed by providers within a provider specialty identified and/or correlating to the request. Furthermore, identifying one or more providers that satisfy the request may optionally include identifying differentiating services from the identified services. Moreover, identifying one or more providers that satisfy the request may optionally include curating a list of differentiating services. Additionally, identifying one or more providers that satisfy the request may optionally include selecting a number of differentiating service to utilize in analysis. Also, identifying one or more providers that satisfy the request may optionally include mapping the selected differentiating services to claims data. Identifying one or more providers that satisfy the request may further optionally include analyzing practices of providers identified within the identified specialty. Furthermore, identifying one or more providers that satisfy the request may optionally include generating a distribution of a volume of the differentiating service provided within a given market within a given period of time. Moreover, identifying one or more providers that satisfy the request may optionally include assigning each provider within the given market a percentage ranking. Additionally, identifying one or more providers that satisfy the request may optionally include determining a threshold in a curve defined by the distribution of the volume of the differentiating service provided within a given market as qualifying a provider as a subspecialist and/or specialist for providing that differentiating service. Also, identifying one or more providers that satisfy the request may optionally include classifying providers meeting the threshold as a subspecialist and/or specialist for performing the differentiating service. Identifying one or more providers that satisfy the request may also optionally include generating one or more searchable directories designating providers as specialists and/or subspecialists based on the assigned classifiers. Act 406 may include any of the actions described above in regard to acts 202-238 of FIG. 2A.

In some embodiments, identifying one or more providers that satisfy the request may not include acts 202-238 of FIG. 2A. Rather, in some embodiments, acts 202-238 may have been performed previously. In other words, receiving the request from the client device 102 may not initiate acts 202-238 in every embodiment. Regardless, identifying one or more providers may include querying the generated searchable directories (e.g., service-specific directories) generated by the provider classification system 104 to identify one or more providers matching the condition and/or service and the market identified in the request, as shown in act 408 of FIG. 4. In some embodiments, the provider classification system 104 may query the searchable directories based on an identified service, a specialty associated with the service, a specialty based on an identified condition, or any other data provided in the request.

In response to identifying one or more providers matching the request, the provider classification system 104 may optionally curate the identified one or more providers, as shown in act 410 of FIG. 4. For instance, the provider classification system 104 may curate the identified one or more providers via any of the methods described in U.S. application Ser. No. 16/584,044, to Stein, filed Aug. 22, 2019.

Upon identifying the one or more providers matching the request or curating the identified one or more providers, the provider classification system 104 may generate a list including the identified or curated one or more providers, as shown in act 412 of FIG. 4. For instance, the provider classification system 104 may generate data packets including a list of the identified or curated one or more providers. The list may include contact information (e.g., address, phone number, website, etc.) of the providers.

Upon generating the list of identified one or more providers, the provider classification system 104 may provide the list to the client device 102 for display within the application 112 of the client device 102, as shown in act 414 of FIG. 4. A provider classification system 104 may provide the list of identified one or more providers to the client device via any of the networks described above in regard to FIG. 1.

In response to receiving the report, the application 112 and/or client device 102 displays the list of identified one or more providers, as shown in act 416 of FIG. 4. For example, in some embodiments, the application 112 of the client device 102 may display the list of identified one or more providers within the application 112 within one or more graphical user interfaces. Additionally, in one or more embodiments, the application 112 of the client device 102 may display the list of identified one or more providers as a notification within the application 112. Alternatively, the client device 102 may display the list of identified one or more providers (e.g., a notification) via the operating system of the client device 102 (e.g., as a push notification).

In view of the foregoing, the provider classification system 104 may provide several advantages. For instance, the provider classification system 104 may remove a need to visit multiple doctors while trying to find a specialist that actually treats a specific condition (e.g., a rare condition) and obtaining a referral. Furthermore, the provider classification system 104 may provide more accurate data to a user in comparison to what a user can obtain on their own. For instance, while a provider may claim to offer a service (e.g., claim to offer service on the provider's website), the provider classification system 104 of the present disclosure may provide a list of providers to a user where in the providers in the list actually do perform the service, and the list of providers may include the highest ranked (e.g., curated) providers within a given market.

Moreover, as will be recognized by one of ordinary skull in the art, the provider classification system 104 may identify subspecialists not previously defined (e.g., one or more providers that provide a unique set of services not previously identified as a subspecialty).

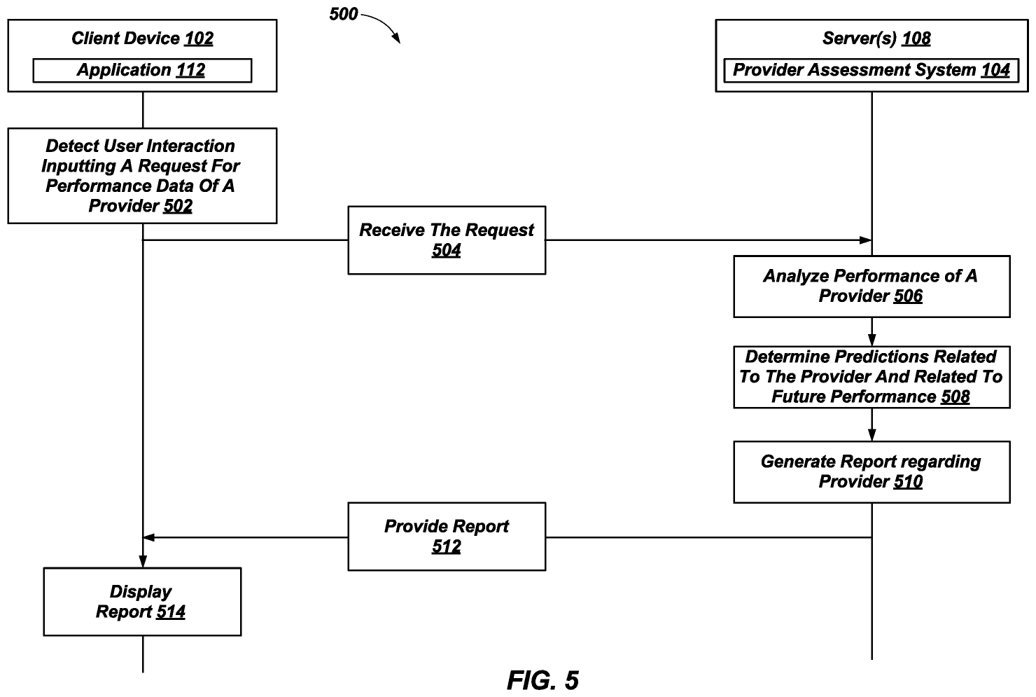

FIG. 5 illustrates a sequence-flow diagram 500 showing various acts of a client device 102 and a provider classification system 104, in accordance with various embodiments of facilitating communications between client devices and the provider classification system 104. In particular, FIG. 5 depicts a method of obtaining performance data related to providers informed by the denominator population described above in regard to FIGS. 2A and 2B. The client device 102 and the provider classification system 104 shown in FIG. 5 may be example embodiments of the client device 102 and the provider determination system(s) 104 described herein.

As shown in act 502 of FIG. 5, in some embodiments, the application 112 and/or client device 102 detects a user interaction inputting a request for performance data (e.g., scores) of a provider. For example, the application 112 of the client device 102 detects a user interaction inputting a provider name and/or selecting an option to receive provider scores of the provider within the application 112 in response to the prompt. For instance, the detected user interaction may be made via graphical user interface.

In response to the client device 102 detecting a user interaction inputting a request for performance data (e.g., scores) of a provider, a provider classification system 104 receives the request from the client device 102, as shown in act 504 of FIG. 5. For instance, the provider classification system 104 may receive a data package including the request for provider performance data. In some embodiments, a provider classification system 104 may receive a data package via any of the networks described above in regard to FIG. 1.

Upon receiving the request for performance data of a provider, a provider classification system 104 analyzes a performance of the provider, as shown in act 506 of FIG. 5. Analyzing the performance of the provider may include performing any of the acts described above in regard to FIGS. 2A-4 and any of the acts described in U.S. application Ser. No. 16/584,044, to Stein, filed Aug. 22, 2019 to determine composites performance scores for the provider. For example, the provider classification system 104 may analyze claims data via performance measures via any of the manners described herein to determine composite performances scores for the provider at the domain level. In some embodiments, a provider classification system 104 may determine composite scores of the provider relative to other providers within a denominator population determined in FIGS. 2A and 2B. For instance, the denominator population may represent the provider's peers.

Furthermore, in some embodiments, a provider classification system 104 determines predictions related to the provider and related to future performance of the provider, as shown in act 508 of FIG. 5. For instance, the provider classification system 104 may apply any of the prediction models described in U.S. application Ser. No. 16/584,044, to Stein, filed Aug. 22, 2019 to measures scores and/or claims data to generate model-predicted measure scores and model-predicted data regarding provider performance.

Additionally, a provider classification system 104 may generate a report including the determined performance scores of the provider and any predicted behaviors and/or predicted performance scores of the provider, as shown in act 510 of FIG. 5. For instance, the report may include any of the performance scores described above, and the report may include indications on how a provider may act (i.e., behave) in the future. Furthermore, the report may show the provider relative to the denominator population determined in FIGS. 2A and 2B.

Upon generating the report, a provider classification system 104 may provide the report to the client device 102 for display within the application 112 of the client device 102, as shown in act 512 of FIG. 5. A provider classification system 104 may provide the report to the client device via any of the networks described above in regard to FIG. 1.

In response to receiving the report, the application 112 and/or client device 102 displays the report, as shown in act 514 of FIG. 5. For example, in some embodiments, the application 112 of the client device 102 may display the report within the application 112 within GUIs. Additionally, in one or more embodiments, the application 112 of the client device 102 may display the report as a notification within the application 112. Alternatively, the client device 102 may display the report (e.g., a notification) via the operating system of the client device 102 (e.g., as a push notification).

Referring to acts 502-514 together, a provider classification system 104 of the present disclosure may enable a user to view performance scores of a provider representing past performance relative to the denominator population (e.g., provider's peers), and a provider classification system 104 of the present disclosure may enable a user to obtain and view data including predictions on how a provider will behave in the future given certain scenarios (e.g., key-clinical decision points).

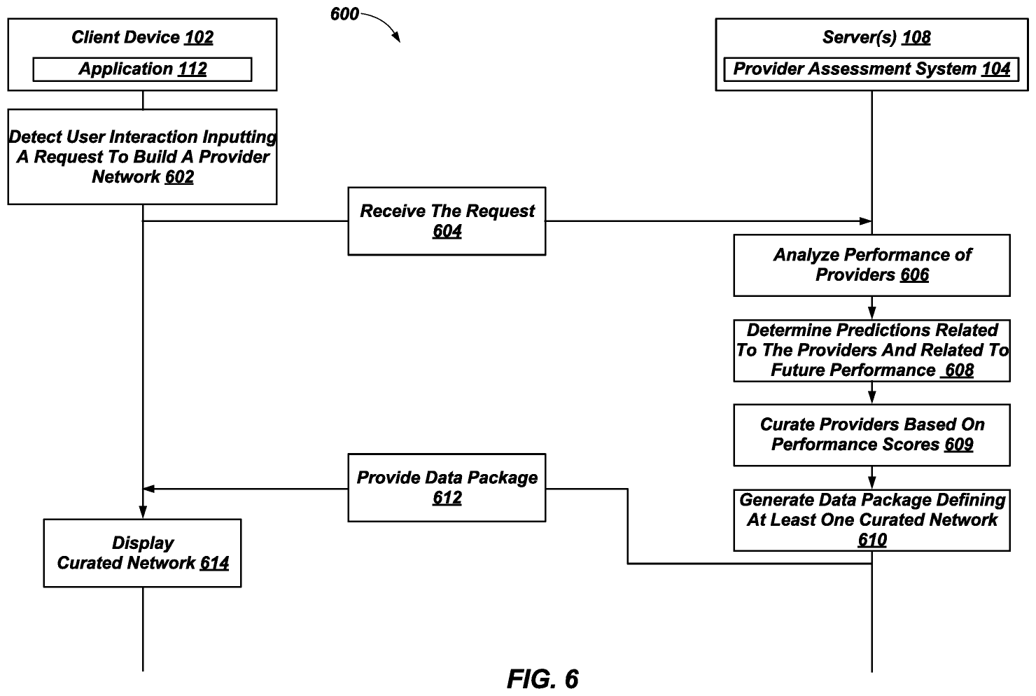

FIG. 6 illustrates a sequence-flow diagram 600 showing various acts of a client device 102 and a provider classification system 104, in accordance with various embodiments of facilitating communications between client devices and the provider classification system 104. The client device 102 and the provider classification system 104 shown in FIG. 6 may be example embodiments of the client device 102 and the provider determination system(s) 104 described herein.

As shown in act 602 of FIG. 6, in some embodiments, the application 112 and/or client device 102 detects a user interaction inputting a request to build a provider network within a given region and/or across one or more specialties. The user interaction may include any of the user interactions described above in regard to FIG. 4. For instance, the request may be made via interactions with GUIs.

In response to the client device 102 detecting a user interaction inputting a request to build a provider network, a provider classification system 104 receives the request from the client device, as shown in act 604 of FIG. 6. For instance, the provider classification system 104 may receive a data package including the request to build a provider network. In some embodiments, a provider classification system 104 may receive the data package via any of the networks described above in regard to FIG. 1.

Upon receiving the request to build a provider network, a provider classification system 104 analyzes the performances of a denominator population of providers within the selected region and/or across the selected specialties, as shown in act 606 of FIG. 6. For instance, the provider classification system 104 may analyze the performance of the denominator population within a selected region and/or the selected specialties, where the denominator population is determined via the manners described above in regard to FIGS. 2A and 2B. Analyzing the performance of the plurality of providers may include performing any of the analysis described above in regard to FIGS. 2A-5 to determine the denominator population and the composites performance scores for each of the providers of the denominator population.

Furthermore, in some embodiments, a provider classification system 104 determines predictions related to each provider of the denominator population within the selected region and/or across the selected specialties and related to future performance of the each provider providers within the selected region and across selected specialties, as shown in act 608 of FIG. 6. For instance, the provider classification system 104 may apply any of the prediction models described above in regard to FIG. 5 to generate model-predicted measure scores and model-predicted data regarding provider performance of each provider of the denominator population within the selected region and across selected specialties.

Moreover, upon determining performance scores and/or performance predictions for the providers within the selected region and/or across the selected specialties, a provider classification system 104 curates the providers of the denominator population within the selected region and across selected specialties, as shown in act 609 of FIG. 6. For instance, a provider classification system 104 may rank the providers based on the composite performance scores of the providers within the selected region and/or across the selected specialties.

Additionally, a provider classification system 104 may generate a data package defining at least one curated network including providers of the denominator population from the selected region and/or across the selected specialties, as shown in act 610 of FIG. 6. For instance, the at least one curated network may include a certain number (e.g., a list) of the best performing providers from the denominator population, according to the determined performance scores of the providers, and within the selected region and/or across the selected specialties. In some embodiments, the provider classification system 104 may define a plurality of curated networks based the determined performance scores of the providers of the denominator population within the selected region and/or across the selected specialties. For example, the provider classification system 104 may define a plurality of curated networks, each curated network being specific to a particular specialty, sub-region, conditions, etc. Furthermore, in some embodiments, provider classification system 104 may define a plurality of curated networks curated by differing performances scores. For example, a first curated network may be curated based on appropriateness scores, a second curated network may be curated based on effectiveness scores, and a third curated network may be curated based on cost scores.

Upon defining the at least one curated network, a provider classification system 104 may provide the data package defining the at least one curated network to the client device 102 for displaying data (e.g., the list) regarding the at least one curated network within the application 112 of the client device 102, as shown in act 612 of FIG. 6. A provider classification system 104 may provide the data package to the client device 102 via any of the networks described above in regard to FIG. 1.

In response to receiving the data package, the application 112 and/or client device 102 displays the at least one curated network, as shown in act 614 of FIG. 6. For example, in some embodiments, the application 112 of the client device 102 may display a list of the providers within the at least one curated network, and the performance scores associated with each provider within the list within the application 112 within GUIs. Additionally, in one or more embodiments, the application 112 of the client device 102 may display the at least one curated network as a notification within the application 112. Alternatively, the client device 102 may display the at least one curated network (e.g., a notification) via the operating system of the client device 102 (e.g., as a push notification).

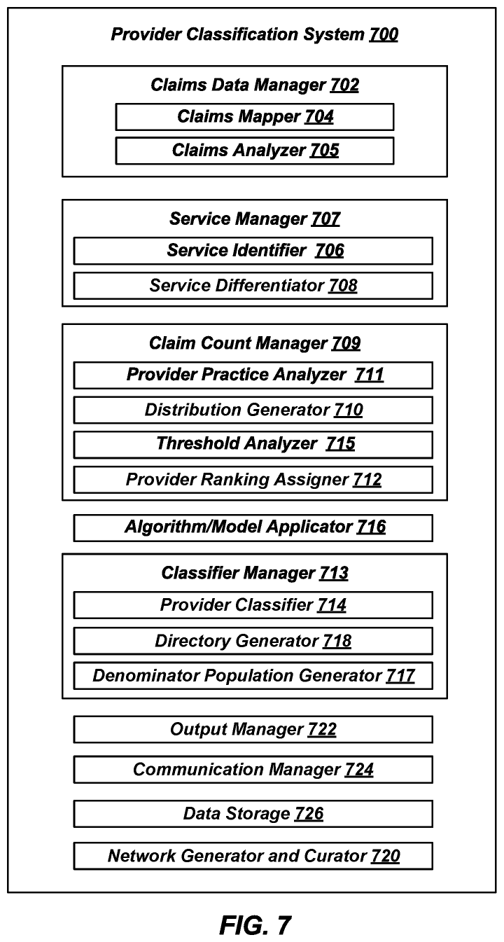

FIG. 7 illustrates a detailed schematic diagram of a provider classification system 700 according to one or more embodiments of the present disclosure.

The provider classification system 700 can be implemented using a computing device including at least one processor executing instructions that cause the provider classification system 700 to perform the processes described herein. In some embodiments, the provider classification system 700 can all be implemented by a single server device, or across multiple server devices. Additionally or alternatively, a combination of one or more server devices and one or more client devices can implement the provider classification system 700. Furthermore, in one embodiment, provider classification system 700 can comprise hardware, such as a special-purpose processing device to perform a certain function. Additionally or alternatively, the provider classification system 700 can comprise a combination of computer-executable instructions and hardware.

In some embodiments, the provider classification system 700 may include a claims data manager 702. The claims data manager 702 may manage claims data received from a third-party system. Furthermore, the claims data manager 702 may provide the claims data to other elements of the provider classification system 700. In one or more embodiments, the claims data manager 702 may include a claims mapper 704 and a claims analyzer 705. In some embodiments, the claims mapper 704 may map differentiating services to claims data via any of the manners described above in regard to FIG. 2A. Furthermore, the claims analyzer may analyze claims data via any of the manners described in regard to FIGS. 2A and 2B.

In one or more embodiments, the provider classification system 700 may further include a service manager 707 that includes service identifier 706 and a service differentiator 708. In some embodiments, the service identifier 706 may identify services typically performed within a given specialty via any of the manners described above in regard to FIG. 2A. The service differentiator 708 may identify differentiating services via any of the manners described above in regard to FIG. 2A.

In some embodiments, the provider classification system 700 may also include a claim count manager 709. The claim count manager 709 may include provider practice analyzer 711, a distribution generator 710, a threshold analyzer 715, and provider ranking assigner 712. The provider practice analyzer 711 may analyze practices of providers via any of the manners described above in regard to FIG. 2A. Furthermore, the distribution generator may generate a distribution via any of the manners described above in regard to FIG. 2A. In some embodiments, the provider classification system 700 may also include an algorithm/model applicator 716. In one or more embodiments, the algorithm/model applicator 716 may apply statistical models, predictive models, principle components analysis, and/or machine learning models to determined identified services and/or data via any of the manners described above in regard to FIGS. 2A and 2B.

In one or more embodiments, the provider classification system 700 may also include a classifier manager 713. The classifier manager 713 may include a provider classifier 714, a directory generator 718, and a denominator population generator 717. The classifier manager 713 may classify providers, the directory generator 716 may generate directories, and the denominator population generator 717 may define a denominator population via any of the manners described above in regard to FIGS. 2A and 2B. Additionally, the provider classification system 700 may further include a network generator and curator 720. The network generator and curator 720 may generate networks and may curate networks via any of the manners described above in regard FIGS. 4-6.

Furthermore, the provider classification system 700 may include an output manager 722 and a communication manager 724. In some embodiments, the output manager 722 may output reports, classifiers, directories, curated networks, built networks, etc. to an application (e.g., tool) of the provider classification system 700 via any of the manners described above in regard to FIGS. 2A-6. Moreover, the communication manager 724 may enable and may manage communication between the provider classification system 700, third-party systems, and client devices via any of the manners described herein.

The provider classification system 700 may also include a data storage 726 (i.e., database) in which the provider classification system 700 may store classification data, service data, data, provider data, performance scores, network definitions, region data, etc.

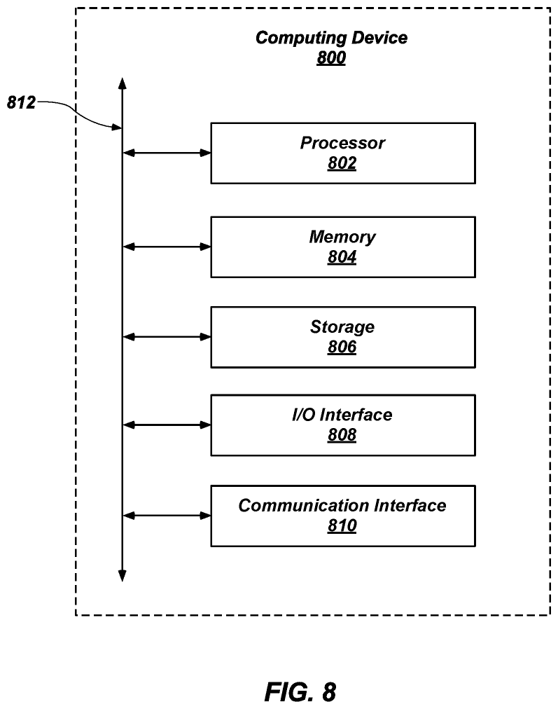

FIG. 8 is a block diagram of an exemplary computing device 800 that may be utilized as a client device (e.g., client device 102) and/or a provider classification system 104 (e.g., provider classification system 104) that may be configured to perform one or more of the processes described above. One will appreciate that one or more computing devices may implement the computing device 800. The computing device 800 can comprise a processor 802, a memory 804, a storage device 806, an I/O interface 808, and a communication interface 810, which may be communicatively coupled by way of a communication infrastructure 812. While an exemplary computing device is shown in FIG. 8, the components illustrated in FIG. 8 are not intended to be limiting. Additional or alternative components may be used in other embodiments. Furthermore, in certain embodiments, the computing device 800 can include fewer components than those shown in FIG. 8. Components of the computing device 800 shown in FIG. 8 will now be described in additional detail.

In one or more embodiments, the processor 802 includes hardware for executing instructions, such as those making up a computer program. As an example and not by way of limitation, to execute instructions, the processor 802 may retrieve (or fetch) the instructions from an internal register, an internal cache, the memory 804, or the storage device 806 and decode and execute them. In one or more embodiments, the processor 802 may include one or more internal caches for data, instructions, or addresses. As an example and not by way of limitation, the processor 802 may include one or more instruction caches, one or more data caches, and one or more translation lookaside buffers (TLBs). Instructions in the instruction caches may be copies of instructions in the memory 804 or the storage 806.

The memory 804 may be used for storing data, metadata, and programs for execution by the processor(s). The memory 804 may include one or more of volatile and non-volatile memories, such as Random Access Memory (“RAM”), Read-Only Memory (“ROM”), a solid state disk (“SSD”), Flash memory, Phase Change Memory (“PCM”), or other types of data storage. The memory 804 may be internal or distributed memory.

The storage device 806 includes storage for storing data or instructions. As an example and not by way of limitation, storage device 806 can comprise a non-transitory storage medium described above. The storage device 806 may include a hard disk drive (HDD), a floppy disk drive, flash memory, an optical disc, a magneto-optical disc, magnetic tape, or a Universal Serial Bus (USB) drive or a combination of two or more of these. The storage device 806 may include removable or non-removable (or fixed) media, where appropriate. The storage device 806 may be internal or external to the computing device 800. In one or more embodiments, the storage device 806 is non-volatile, solid-state memory. In other embodiments, the storage device 806 includes read-only memory (ROM). Where appropriate, this ROM may be mask programmed ROM, programmable ROM (PROM), erasable PROM (EPROM), electrically erasable PROM (EEPROM), electrically alterable ROM (EAROM), or flash memory or a combination of two or more of these.

The I/O interface 808 allows a user to provide input to, receive output from, and otherwise transfer data to and receive data from computing device 800. The I/O interface 808 may include a mouse, a keypad or a keyboard, a touch screen, a camera, an optical scanner, network interface, modem, other known I/O devices or a combination of such I/O interfaces. The I/O interface 808 may include one or more devices for presenting output to a user, including, but not limited to, a graphics engine, a display (e.g., a display screen), one or more output drivers (e.g., display drivers), one or more audio speakers, and one or more audio drivers. In certain embodiments, the I/O interface 808 is configured to provide graphical data to a display for presentation to a user. The graphical data may be representative of one or more graphical user interfaces and/or any other graphical content as may serve a particular implementation.

The communication interface 810 can include hardware, software, or both. In any event, the communication interface 810 can provide one or more interfaces for communication (such as, for example, packet-based communication) between the computing device 800 and one or more other computing devices or networks. As an example and not by way of limitation, the communication interface 810 may include a network interface controller (NIC) or network adapter for communicating with an Ethernet or other wire-based network or a wireless NIC (WNIC) or wireless adapter for communicating with a wireless network, such as a WI-FI.

Additionally or alternatively, the communication interface 810 may facilitate communications with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, the communication interface 810 may facilitate communications with a wireless PAN (WPAN) (such as, for example, a BLUETOOTH®WPAN), a WI-FI network, a WI-MAX network, a cellular telephone network (such as, for example, a Global System for Mobile Communications (GSM) network), or other suitable wireless network or a combination thereof.

Additionally, the communication interface 810 may facilitate communications various communication protocols. Examples of communication protocols that may be used include, but are not limited to, data transmission media, communications devices, Transmission Control Protocol (“TCP”), Internet Protocol (“IP”), File Transfer Protocol (“FTP”), Telnet, Hypertext Transfer Protocol (“HTTP”), Hypertext Transfer Protocol Secure (“HTTPS”), Session Initiation Protocol (“SIP”), Simple Object Access Protocol (“SOAP”), Extensible Mark-up Language (“XML”) and variations thereof, Simple Mail Transfer Protocol (“SMTP”), Real-Time Transport Protocol (“RTP”), User Datagram Protocol (“UDP”), Global System for Mobile Communications (“GSM”) technologies, Code Division Multiple Access (“CDMA”) technologies, Time Division Multiple Access (“TDMA”) technologies, Short Message Service (“SMS”), Multimedia Message Service (“MMS”), radio frequency (“RF”) signaling technologies, Long Term Evolution (“LTE”) technologies, wireless communication technologies, in-band and out-of-band signaling technologies, and other suitable communications networks and technologies.

The communication infrastructure 812 may include hardware, software, or both that couples components of the computing device 800 to each other. As an example and not by way of limitation, the communication infrastructure 812 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination thereof.

The embodiments of the disclosure described above and illustrated in the accompanying drawing figures do not limit the scope of the invention, since these embodiments are merely examples of embodiments of the invention, which is defined by the appended claims and their legal equivalents. Any equivalent embodiments are intended to be within the scope of this invention. Indeed, various modifications of the present disclosure, in addition to those shown and described herein, such as alternative useful combinations of the content features described, may become apparent to those skilled in the art from the description. Such modifications and embodiments are also intended to fall within the scope of the appended claims and legal equivalents.

1. A method of classifying a provider, comprising:
analyzing, via at least one processor, claims data from providers within a selected specialty;
identifying, at least partially via the analysis of the claims data, differentiating services from services performed by providers within the selected specialty and within a selected market;
curating a list of differentiating services from the identified differentiating services;
generating a data package including the list of differentiating services;
analyzing practices of providers within the selected specialty and the selected market representing in the claims data;
based on the analysis of the practices of providers, generating a data package including a distribution of a volume of at least one differentiating service of the list of differentiating services and performed by providers within the selected specialty and the selected market;
determining a threshold within the distribution of the volume of the at least one differentiating service for classifying a provider as a specialist in performing the at least one differentiating service;
generating a label representing a classification of providers as performing the at least one differentiating service based on the threshold; and
classifying one or more providers within the selected specialty and the selected as a specialist in performing the at least one differentiating service based on the threshold and by assigning the generated label to the one or more providers.

2. The method of claim 1, further comprising generating one or more data packages representing service or specialty-specific directories of the classified one or more providers.

3. The method of claim 1, further comprising:
performing one of adding the generating label to a directory already including the one or more providers or comparing the generated label to labels of the one or more providers with a directory; and
based on the addition or comparison, determining whether to keep or remove the one or more providers from the directory.

4. The method of claim 1, further comprising mapping the at least one differentiating service of the list of differentiating services to one or more coding fields of claims data, wherein mapping at least one differentiating service to one or more coding fields of claims data comprises mapping at least one differentiating service to coding fields of claims data via one or more of field mapping or field value mapping methodologies.

5. The method of claim 4, wherein analyzing the practices of providers within the selected specialty and the selected market comprises analyzing claims data utilizing mapping data from the mapped at least one differentiating service.

6. The method of claim 1, further comprising assigning each provider within the selected market a percentage ranking within the generating distribution of a volume of the at least one differentiating service.

7. The method of claim 1, further comprising determining a denominator population of providers qualifying as specialists in performing the at least one differentiating service within the selected market.

8. The method of claim 7, further comprising determining performance scores of the providers of the denominator population of providers.

9. The method of claim 8, further comprising curating a network of providers based on the performance scores of the providers of the denominator population of providers.

10. The method of claim 1, wherein classifying one or more providers as a specialist comprises utilizing machine-learning techniques to determine the labels representing classifications for classifying the one or more providers.

11. A system comprising:
at least one processor; and
at least one non-transitory computer-readable storage medium storing instructions thereon that, when executed by the at least one processor, cause the system to:
curate a list of differentiating services performed by providers within a selected specialty and a selected market;
analyze practices of providers within the selected specialty and the selected market;
based on the analysis of practices of providers, generate a distribution of a volume of at least one differentiating service performed by providers within the selected specialty and the selected market;
determine a threshold within the distribution of the volume of the at least one differentiating service for qualifying a provider as a specialist in performing the at least one differentiating service;
assign each provider within the selected market a percentage ranking within the distribution of a volume of the at least one differentiating service;
generate classifiers for one or more providers, each classifier including an indication of the threshold, the provider's percentage ranking, and the specialty in performing the at least one differentiating service; and
assign the classifiers to respective one or more providers.

12. The system of claim 11, further comprising instructions that, when executed by the by the at least one processor, cause the system to generate one or more service or specialty-specific directories of the classified one or more providers.

13. The system of claim 11, wherein analyzing practices of providers within the selected specialty and the selected market comprises analyzing practices of providers within a specific period of time.

14. The system of claim 11, further comprising instructions that, when executed by the by the at least one processor, cause the system to map the at least one differentiating service to coding fields of claims data via one or more of field mapping or field value mapping methodologies.

15. The system of claim 11, wherein analyzing practices of providers within the selected specialty and the selected market comprises analyzing claims data utilizing mapping data from the mapped at least one differentiating service.

16. The system of claim 11, further comprising instructions that, when executed by the by the at least one processor, cause the system to determine a denominator population of providers being assigned classifiers as specialists in performing the at least one differentiating service within the selected market.

17. The system of claim 11, further comprising instructions that, when executed by the by the at least one processor, cause the system to determine performance scores of the providers of the denominator population of providers.

18. The system of claim 17, further comprising instructions that, when executed by the by the at least one processor, cause the system to curate a network of providers based on the performance scores of the providers of the denominator population of providers.

19. A method of classifying a provider, comprising:
analyzing practices of providers within a selected specialty and a selected market by analyzing claims data;
based on the analysis of practices of providers, generating a data package representing a distribution of a volume of at least one differentiating service performed by providers within the selected specialty and the selected market;
determining a threshold within the distribution of the volume of the at least one differentiating service for classifying a provider as a specialist in performing the at least one differentiating service; and
generating a label representing a classification of providers as performing the at least one differentiating service based on the determined threshold; and
classifying one or more providers as a specialist in performing the at least one differentiating service based on the threshold and by assigning the generated label to the one or more providers.

20. The method of claim 1, further comprising assigning each provider within the selected market a percentage ranking within the distribution of a volume of the at least one differentiating service.
